(19) United States
(12) Patent Application Publication
(10) Pub. No.: US 2023/0101709 A1
(43) Pub. Date: Mar. 30, 2023

(54) PIPETTABLE REPLACEMENT FLUID FOR MIMICKING PIPETTABILITY OF TARGET FLUID AND RELATED METHOD

(71) Applicant: ARTEL, INC., Westbrook, ME (US)

(72) Inventors: John Thomas Bradshaw, Gorham, ME (US); Rachel Parshley, Portland, ME (US); Heather Macken, Sebago, ME (US); Michael Grimaldi, Portland, ME (US); Richard Curtis, Gorham, ME (US)

(21) Appl. No.: 17/665,514

(22) Filed: Feb. 5, 2022

Related U.S. Application Data
(60) Provisional application No. 63/250,983, filed on Sep. 30, 2021.

(51) Int. Cl. G01N 33/96

(52) U.S. Cl. CPC G01N 33/96; G01N 2496/25

(57) ABSTRACT

A replacement fluid suitable to replace a target fluid, wherein the replacement fluid is different from the target fluid. The replacement fluid has pipetting characteristics substantially equivalent to the target fluid, wherein for a desired pipetted volume of the target fluid delivered by a pipette, a pipetted volume of the replacement fluid delivered by the pipette is substantially equivalent to the desired pipetted volume of the target fluid. The replacement fluid is a primary equivalent fluid having substantially similar pipetting characteristics. One or more additives may be added to the primary equivalent fluid. The one or more additives may include pipettability modifying additives and non pipettability modifying additives.

### BACKGROUND OF THE INVENTION

### 1. Field of the Invention

The present invention relates to fluids selected to mimic pipetting characteristics of target fluids so that the fluids can function as replacement fluids for the target fluids. In particular, the present invention relates to fluids having rheological and other characteristics that enable the fluids to substantially mimic the pipettability of the target fluids.

### 2. Description of the Prior Art

Pipettes are used to transfer fluids of interest from a source location to a target location. Pipettes may be operated manually or automatically, such as with an automated liquid handler. Pipettes may be used singly or grouped together. The fluids that are analyzed to gather information contained therein are referred to herein as “target fluids.” Fluids transferred with pipettes can be of all sorts, with varying transfer volume size and accuracy requirements dependent on the availability of the target fluid at the source, the type of target fluid, the target location conditions, and the target fluid analysis to be conducted, to name a few. The analysis of the target fluid may be greatly impacted by the accuracy of the volume of the target fluid transferred with the pipette. As a result, achieving optimal analysis performance from pipette usage is more difficult than users may expect—particularly when the target fluid volume transferred is small or when the physical and chemical properties of various target fluids used within an assay are significantly different from each other.

Pipetting characteristics of fluids are dependent upon physical characteristics of those fluids, including density, viscosity, surface tension and other properties. These characteristics are determined by the chemical components in those fluids. The accurate and repeatable delivery of specified volumes of fluid from pipetting devices depends in part on those characteristics. For example, pipetting settings selected to deliver a certain volume of serum manually or automatically are different than those needed to deliver the same volume of pure water. Conducting pipetting operations with unreliable target fluid characteristics can result in over-delivered, under-delivered, or inconsistently-delivered target fluid volumes in assays used for clinical diagnostics, pharmaceutical development, and other areas of life sciences and other analysis environments where quality assurance is of most importance.

Serum is just one example of a target fluid that is often pipetted. It is commonly used as test media in pharmaceutical, clinical, and other life-science laboratory testing. It is a critical component in many fields such as cell analysis, and biological test assays. Serum from one animal type may be significantly different from another animal type. Each sera contains various components that change the density, viscosity, surface tension, shear rate and contact angle, which in turn change the pipetting characteristics of the sera, as compared to water. Each sera may also contain components that change other physical and chemical properties, such as the propensity of depositing an adsorbed layer of molecules inside the pipette tip, which may also impact pipetting characteristics. If a significant adlayer of molecules form on the inside of the pipette tip, the result could be a change in the shear rate experienced by the liquid due to a smaller orifice. This adlayer can also change the surface chemical properties of the inside pipette tip from hydrophobic to hydrophilic, which in turn may result in a thicker sheet of liquid adhering to the inner pipette wall. These parameters can result in different pipetting characteristics and may need to be mimicked to adequately model a solution of interest. Different solution additives may be used to cause the same degree of molecular adsorption as in the solution of interest. The different characteristics of one type of serum require different liquid handling steps to achieve the same pipetted volume as compared to another type of serum (or as compared to water). If a user does not account for the need for different liquid handling steps, he may be grossly over dispensing or under dispensing the serum he is using without knowing it. He may also be dispensing a variable amount of serum without knowing it. An over-dispense, an under-dispense or uncontrolled variability in dispense may result in erroneous data and conclusions.

The task of pipetting an accurate volume of fluid reliably and repeatably is not simple, particularly when very small volumes of fluid are involved—on the order of 2 mL or less, and especially for volumes less than 200 μL, for example. When that target fluid is expensive and/or in short supply, any user training, automated dispensing equipment calibration, test protocol optimization, or analysis equipment calibration is also expensive. As a result, there may be a reluctance to conduct such training, optimization or calibration steps, which can lead to inaccuracies and lack of repeatability when necessary fluid dispensing is carried out. There exist some fluids that can be used to conduct training, optimization, and calibration but they do not match the pipetting characteristics of target fluids of interest including the ones that are expensive or are in short supply. These possible replacement fluids can be useful, but because they do not match the pipetting characteristics of target fluids of interest, they also can lead to inaccuracies and lack of repeatability.

There are several parameters that can effect pipetting, including the speed at which a fluid is aspirated into the pipette, the speed with which a fluid is dispensed from the tip, the amount of fluid left inside the tip (as a thin layer coating the inner wall), the insertion depth of the pipette tip into the solution to aspirate, the insertion depth into the solution to dispense, etc. All of these parameters collectively impact the final volume that is ultimately dispensed by the pipette and can be referred to as the pipettability or pipetting characteristics of the fluid. While some parameters are somewhat universal to any solution that is being pipetted, others are strongly effected by the identity of the solution that is being used. For any solution being pipetted, a sufficient replacement fluid must ultimately dispense the same way the target fluid does. Thus, the best measure of how good a replacement fluid is, is in how closely the replacement pipettes a specific volume when it is aspirated and dispense in the exact same manner as the target fluid of interest.

What is needed is a replacement fluid or replacement fluids that substantially match or mimic the pipetting characteristics of a target fluid or target fluids of interest. Further, what is needed is a replacement fluid or replacement fluids that impart pipetting characteristics that substantially mimic a target fluid or target fluids of interest without being unreasonably expensive and are more likely to be supplied than the target fluid or target fluids. Yet further, what is needed is a related method available to pipette users to produce such replacement fluids through identification of those replacement fluid characteristics necessary to enable pipetting in a substantially similar manner as the target fluids of interest that the replacement fluids are intending to mimic.

### SUMMARY OF THE INVENTION

It is an object of the present invention to enable the production of one or more replacement fluids having pipetting characteristics, which may also be referred to herein as “pipettability,” that mimic the pipetting characteristics of one or more target fluids of interest. This object is achieved by defining the replacement fluid rheology that when established produce in the replacement fluid pipetting characteristics that mimic the pipetting characteristics of a target fluid.

The present invention is a replacement fluid and related method for making the replacement fluid wherein the replacement fluid mimics the pipetting characteristics of a target fluid of interest. The replacement fluid is made of one or more components selected to substantially mimic pipettability while being either or both of less expensive and more available than the target fluid being mimicked. Specifically, the replacement fluid mimics the rheology of the target fluid of interest.

Rheology is the study of the deformation and flow of matter, including how materials respond to applied stress and strain. Rheological properties are important in pipetting based on the physical process in which a pipette aspirates and dispenses a fluid. Pipetting is achieved by applying a force to a fluid to cause it to either move into the pipette tip (i.e., aspirate), or to move out of the pipette tip (i.e., dispense). The rheological properties of a fluid dictate the rate at which that fluid will flow into or out of the pipette, as well as how much fluid coats the inside of the pipette tip, etc. Rheological properties include both chemical and physical characteristics and include viscosity, shear rate, surface tension and contact angle of the fluid with respect to a surface material. The viscosity of a material determines how gelatinous, syrupy or viscous that material is and is related to the chemical and physical attractions experienced by the molecules in a fluid. A common comparison to define viscosity is to compare water to honey, with the obvious observation that honey is much thicker or gelatinous than water and flows much slower when poured at room temperature. If honey is heated to near boiling and poured, it flows at nearly the same rate as water, because its viscosity has dropped to nearly the same as water. Thus, viscosity is impacted by the temperature of the material. Applying a force can also impact the viscosity of matter and is defined by a characteristic called shear rate; in some cases, applying force (e.g., shear stress) causes a material's viscosity to increase (i.e., shear thickening) and in other cases applied force causes the material's viscosity to decrease (i.e., shear thinning).

Yet another property is the surface tension of a fluid, which is a measure of the cohesive interactions between the molecules in a fluid specifically at the fluid's surface. The most common example of a fluid with very high surface tension is water wherein the strong cohesion of the water molecules with each other (as opposed to attraction between water molecules and air) results in a skin-like surface that can float more dense materials (e.g., a paper clip). Water's surface tension can be significantly reduced with a drop of dish soap, oil, or other hydrophobic materials. Contact angle is yet another property, which is defined as the angle that is formed between a fluid droplet and a surface it sits on. To envision this, think of a water droplet on a flat plastic sheet. Plastic is hydrophobic, so the water wants to exist in a tight ball and will have a strong radius of curvature as it attempts to minimize how much it has to touch the plastic. This results in a large contact angle (more than 90 degrees). Conversely, water on a flat sheet of clean glass (glass is hydrophilic) will easily spread out, forming a sheet across the glass with a contact angle near zero.

Contact angle is a combination of the physical and chemical properties of the fluid and the physical and chemical properties of the material it is in contact with. Contact angle determines if the fluid wants to stick to the pipette tip and leave behind a thin layer or wants to get off the pipette tip and is thereby easily removed. There are also other properties that loosely relate to rheology but have a direct impact on the pipetting characteristics of a fluid which include the relative density, as well as the chemical interactions of the fluid components with their surroundings (e.g., surface adsorption of molecules onto a surface). For a replacement fluid to substantially mimic the pipetting characteristics of a target fluid of interest, it will need to substantially mimic most of the rheological and other characteristics of that target fluid.

The rheological characteristics that should be substantially matched (or are at least sufficiently comparable) to produce a substantially similar pipettability include relative density, viscosity, shear rate, and surface tension. In addition, to those characteristics of the replacement fluid itself, the pipettability that the replacement fluid needs to match may also be dependent on the contact angle of the replacement fluid in relation to the material of the tip of the pipette. This contact angle is dependent upon both the physical and chemical properties of the fluid and the physical and chemical properties of the pipette tip material. Thus, the contact angle of the replacement fluid with respect to the pipette tip material should be substantially similar to the contact angle of the target fluid of interest with respect to the pipette tip material.

The present invention is a replacement fluid having a combination of the identified rheological properties and other characteristics of the target fluid that results in pipetting characteristics in the replacement fluid that are substantially similar to the pipetting characteristics of the target fluid of interest. The resulting replacement fluid should be capable of delivering a pipetted volume substantially similar to, and within about +/−20%, of the same delivered volume of the target fluid of interest.

Each replacement fluid of the present invention that is a pipetting equivalent of a target fluid of interest includes a primary equivalent fluid and may include one or more other pipettability modifying additives that impart chemical or physical properties to the primary equivalent fluid that render its pipettability substantially the same as that of a target fluid of interest. The primary equivalent fluid is chosen for an approximation of the general chemical and physical properties of the target fluid (e.g., water for aqueous solutions, organic solvent for organic solutions). The at least one or more optional modifying additives are chosen to modify the relative density, the viscosity, the surface tension, the shear rate, and/or the contact angle of the replacement fluid such that the pipetting characteristics are substantially similar to the target fluid. A pipetted volume of the replacement fluid should be within about +/−20% of the solution of interest. The one or more optional modifying additives are chosen to modify at least the viscosity and the surface tension characteristics of the primary equivalent fluid but not limited thereto.

The one or more optional modifying additives may also modify the relative density, shear rate, surface tension, and/or contact angle of the primary equivalent fluid. The primary equivalent fluid and the optional modifying additives are also chosen for relative availability and lower cost in comparison to the target fluid of interest. Further consideration of the properties of the primary equivalent fluid and the one or more modifying additives is their temperature dependence. That is, the effect of temperature on the rheological characteristics of a combination of the primary equivalent fluid and the optional one or more pipetting modifying additives should be substantially equivalent to the temperature dependency of the target fluid of interest.

Yet further consideration of the properties of the primary equivalent fluid and the one or more modifying additives is their dependence on applied force or stress. That is, the effect of applied force of shear stress on the rheological characteristics of the combination of the primary equivalent fluid and the one or more modifying additives should be substantially equivalent to the applied force or shear stress dependency of the target fluid of interest.

Examples of the optional one or more pipettability modifying additives that may be added to the primary equivalent fluid to adjust the rheological and other chemical and physical properties of the primary equivalent fluid include one or more of water, buffering salt, glycerol, sucrose, protein, lipid, surfactant, and soluble polymer. These modifying additives may be added to increase or decrease the relative density of the composition, the surface tension, the viscosity and shear rate, and the contact angle. For example, buffering salt may be added to change the relative density and surface tension of the composition, whereas glycerol, sucrose or protein may be added to change the viscosity of the composition, and whereas lipid or surfactant may be added to change the surface tension and contact angle of the composition with respect to the pipette tip material. Protein and soluble polymer may be added to increase viscosity or to produce an adsorbed layer on the inner pipette wall.

The composition of the invention may further include optional additives which do not affect pipettability (called non-modifying additives herein). The optional non-modifying additives are selected to produce certain characteristics of specific interest without adversely impacting the equivalent rheological mimicking characteristics of the replacement fluid. These non pipetting modifying additives may have different purposes. For example, one or more dyes may be added to the replacement fluid to facilitate pipetting training and/or equipment calibration. The one or more dyes may be absorbance or fluorescence dyes useful for performing spectroscopic measurements to determine the volume of a reagent dispensed. Other additives may also include stabilizing buffers or chelators, and well as preservative agents to extend the shelf-life of the replacement fluid.

An example replacement fluid of the present invention is a replacement fluid that mimics the pipettability of serum. Serum is a complex biological mixture that contains all of the soluble constituents of an animal's blood. Some of the components of serum include dissolved proteins (e.g., hemoglobin, albumin, immunoglobulin, etc.), lipids (e.g., cholesterol, triglycerides, etc.), sugars (e.g., glucose), buffering salts (e.g., sodium, calcium, chloride, potassium, etc.), etc. Each of these components imparts some impact on the pipetting characteristics of serum that, when taken as a whole, define the pipettability of serum. Therefore, a replacement fluid must substantially mimic these pipetting characteristics to be a successful substitute for serum. The different characteristics require different liquid handling steps for serum as compared to the liquid handling steps needed for water. If a user does not account for the need for different liquid handling steps, he may be grossly over dispensing or under dispensing the serum without knowing it. An over-dispense results in wasting expensive reagent. An under-dispense my result in inaccurate dilutions, which may result in erroneous data and conclusions. Depending on the animal source, serum can be very expensive and hard to gather in large quantities, so it is desirable to have available a pipetting equivalent.

Serum is a complex liquid, but not all constituents are needed to substantially impart the necessary pipetting characteristics. A substantial serum mimicking replacement fluid includes water as the primary equivalent fluid, and pipettability modifying additives of potassium hydrogen phthalate (KHP) as a buffering salt, and bovine serum albumin (BSA). The weight percent of BSA in the replacement fluid is in the range of about 0.5%-to-5%. As compared to pure water, the BSA increases the viscosity from 1 centipoise (cP) to above 2.5 cP at 5° C. and with no shear stress applied and can be used to tailor the viscosity to have substantially the same shear rate as a serum of interest. The KHP increases the relative density and viscosity and also stabilizes the BSA protein in solution. The BSA and KHP both significantly decrease the surface tension to between 30-35 mN/m, which is well below the surface tension of water (75 mN/m), and also have an impact on the contact angle. These two components make a serum pipetting replacement fluid equivalent that is much more readily available and substantially less expensive than serum.

The invention is a replacement fluid suitable to replace a target fluid, wherein the replacement fluid is different from the target fluid. The replacement fluid includes a fluid having pipetting characteristics substantially equivalent to the target fluid, wherein for a desired pipetted volume of the target fluid delivered by a pipette, a pipetted volume of the replacement fluid delivered by the pipette is substantially similar to the desired pipetted volume of the target fluid. The pipetted volume of the replacement fluid may be within about +/−20 of the desired pipetted volume of the target fluid. The replacement fluid has rheological and other properties substantially equivalent to rheological and other properties of the target fluid. The rheological and other properties include viscosity, shear rate, surface tension, and contact angle. The replacement fluid may include a primary equivalent fluid and one or more additives. The one or more additives may include an additive that adsorbs to a surface of a pipette tip. The one or more additives may include one or more pipetting-modifying additives selected to modify one or more of the rheological and other properties of the primary equivalent fluid. The one or more pipetting-modifying additives may be selected from the group consisting of water, a water-soluble polymer, buffering salt, glycerol, sucrose, protein, lipid, and surfactant. The one or more additives may include one or more non-pipetting-modifying additives selected not to modify one or more of the rheological properties of the primary equivalent fluid. The one or more non-pipetting-modifying additives may be selected from the group consisting of stabilizing buffer, chelator, and preservative agent. In an embodiment, the replacement fluid is water and one of the one or more pipetting-modifying additives is bovine serum albumin.

Further, the invention is a replacement fluid suitable to replace a target fluid to be analyzed, wherein the replacement fluid is different from the target fluid. That replacement fluid includes a fluid having pipetting characteristics substantially equivalent to the target fluid, wherein for a desired pipetted volume of the target fluid delivered by a pipette, a pipetted volume of the replacement fluid delivered by the pipette is substantially similar to the desired pipetted volume of the target fluid, and one or more analysis-aiding components, wherein the one or more analysis-aiding components are selected to enable analysis of the replacement fluid as though it were the target fluid. The one or more analysis-aiding components may be one or more absorbance or fluorescence dyes selected to enable spectroscopic analysis of the replacement fluid. The replacement fluid of this embodiment has rheological and other properties substantially equivalent to rheological properties of the target fluid. The rheological and other properties include viscosity, shear rate, surface tension, and contact angle. The replacement fluid may be a primary equivalent fluid and one or more additives. The one or more additives may include one or more pipetting-modifying additives selected to modify one or more of the rheological and other properties of the primary equivalent fluid. The one or more pipetting-modifying additives may be selected from the group consisting of water, buffering salt, glycerol, sucrose, protein, lipid, water-soluble polymer, and surfactant. The one or more additives may include one or more non-pipetting-modifying additives selected not to modify one or more of the rheological properties of the primary equivalent fluid. The one or more non-pipetting-modifying additives may be selected from the group consisting of stabilizing buffer, chelator, and preservative agent. In an embodiment, the replacement fluid may be water and one of the one or more pipetting-modifying additives is bovine serum albumin.

It can be seen that any replacement fluid can be made with selective rheological characteristics mimicking those of a target fluid of interest. The components of that replacement fluid may be chosen based on those equivalent characteristics as well as availability and cost. The invention is one or more replacement fluids with target fluid pipette mimicking characteristics. Those pipette mimicking characteristics are produced by selecting the primary equivalent fluid and, one or more optional pipettability modifying additives to generate substantially equivalent pipetting characteristics.

### BRIEF DESCRIPTION OF THE DRAWINGS

FIG. 1 is a graph showing the viscosity as a function of shear rate for human serum, horse serum, a first example serum substitute (called SerumSub A) and a second example serum substitute (called SerumSum B).

FIG. 2 is a graph showing a comparison of pipettability for serum solutions at 10 μL.

FIG. 3 is a graph showing a comparison of pipettability for serum solutions at 25 μL.

FIG. 4 is a graph showing a comparison of pipettability for serum solutions at 50 μL.

FIG. 5 is a graph showing a comparison of pipettability for serum solutions at 100 μL.

FIG. 6 is a graph showing a comparison of pipettability for serum solutions at 200 μL.

### DETAILED DESCRIPTION OF THE INVENTION

A replacement fluid of the present invention for mimicking the pipetting characteristics of a target fluid of interest is a primary equivalent fluid having substantially similar pipettability without being the target fluid. The primary equivalent fluid may be combined with one or more pipetting modifying additives that impart chemical and physical properties of pipettability interest. There may be additional optional non pipetting modifying additives forming part of the replacement fluid. The modifying additives and the ratio of such additives in the replacement fluid are selected to generate in the replacement fluid a pipettability that is substantially the same as the pipettability of the target fluid of interest.

A first example of a replacement fluid of the present invention is a replacement fluid that mimics the pipettability of serum. A first example of a fluid replacement for serum includes water as the primary equivalent fluid, and about 6 g/L of BSA and about 4 g/L of KHP as pipettability modifying additives. Further non-pipettability modifying additives include about 4 g/L of the chelating agent ethylenediaminetetraacetic acid (EDTA) and about 0.6 g/L of the preservative Mergal (Troy Chemical, part number 100421). Yet further non-pipettability modifying additives include 1.1 g/L of copper chloride dihydrate as one spectroscopic dye, and about 0.33 g/L of Ponceau S as a second spectroscopic dye (the amount of Ponceau S depends on the spectroscopic properties needed). Further additions of hydrochloric acid and/or sodium hydroxide are made to bring the fluid to a pH of 6.

A second example of a fluid replacement for serum includes water as the primary fluid, and about 6 g/L of BSA and about 4 g/L of KHP as pipettability modifying additives. Further non-pipettability modifying additives include about 4 g/L of the chelating agent ethylenediaminetetraacetic acid (EDTA) and about 0.6 g/L of the preservative Mergal (Troy Chemical, part number 100421). Yet further non-pipettability modifying additives include about 1.1 g/L of copper chloride dihydrate as one spectroscopic dye, and about 0.08 g/L of Ponceau S as a second spectroscopic dye (the amount of Ponceau S depends on the spectroscopic properties needed). Further additions of hydrochloric acid and/or sodium hydroxide are made to bring the fluid to a pH of 6.

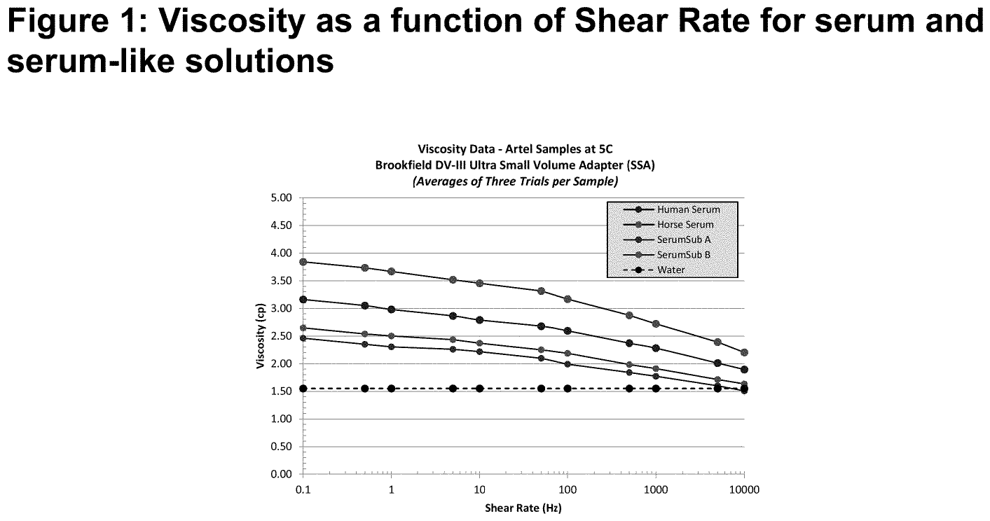

Test results for the first and second examples of a fluid replacement for serum include testing of rheological properties as shown in FIG. 1 and Table 1 (below). FIG. 1 is a graph showing the viscosity as a function of shear rate for human serum, horse serum, a first example serum substitute (called SerumSub A) and a second example serum substitute (called SerumSum B). An important observation from this data is how different the viscosity of water is as compared to the serum and serum-like samples. The viscosity of water remains flat and is not strongly impacted by applied shear stress. However, each serum and each serum-substitute experiences a significant decrease in viscosity as shear stress is applied (shear thinning). This shear thinning results in solution that flows more freely. Addition of the BSA and KHP to water significantly changes the viscosity and viscosity profile as compared to water, making the serum-like replacement fluids behave more like serum than like water.

Table 1 further demonstrates how additives can make rheological properties less like water, and more like serum. Table 1 provides the surface tension of human and horse sera at 59 mN/m and 52 mN/m, respectively, which is considerably lower than the surface tension of water at 75 mN/m. Adding BSA and the other named components results in surface tensions for SerumSub A and SerumSub B of 57 mN/m and 45 mN/m, which are more in line with serum than water.

TABLE 1
Surface tension at 5° C. for human serum, horse serum, a first example
serum-like fluid and a second example serum-like fluid.

| Human | Horse | | | |
|---|---|---|---|---|
| Serum | Serum | SerumSub A | SerumSub B | Water |
| at 5° C. | at 5° C. | at 5° C. | at 5° C. | at 5° C. |
| Surface | Surface | Surface | Surface | Surface |
| Tension | Tension | Tension | Tension | Tension |
| (mN/m) | (mN/m) | (mN/m) | (mN/m) | (mN/m) |
| 58.61 | 51.89 | 56.97 | 45.08 | 74.94 |

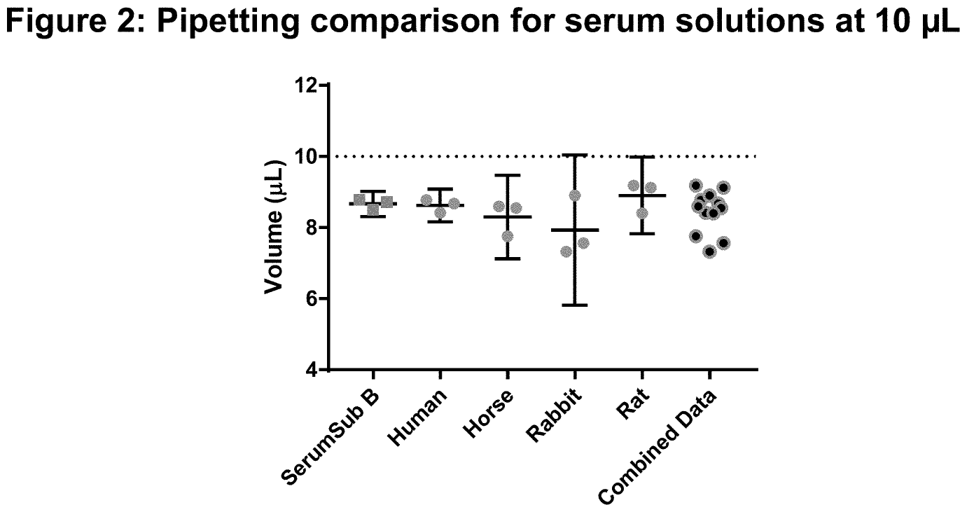

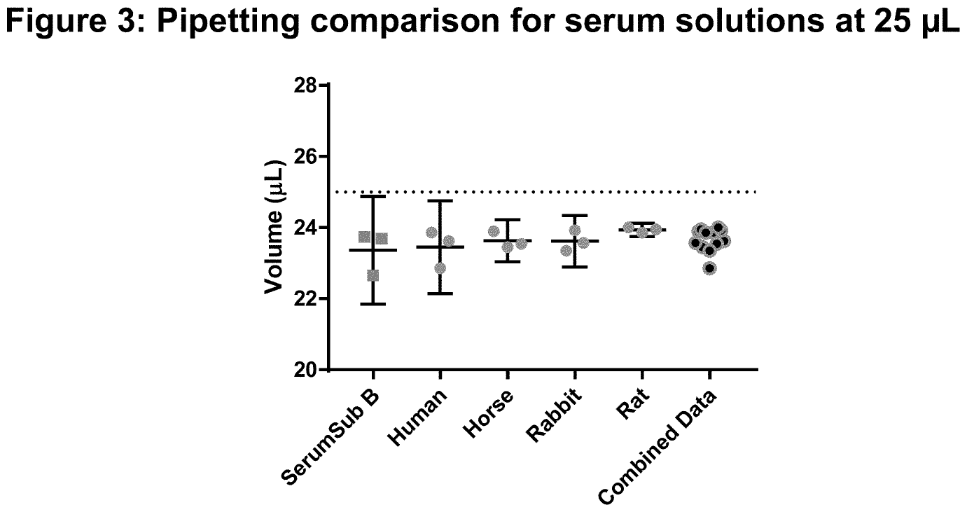

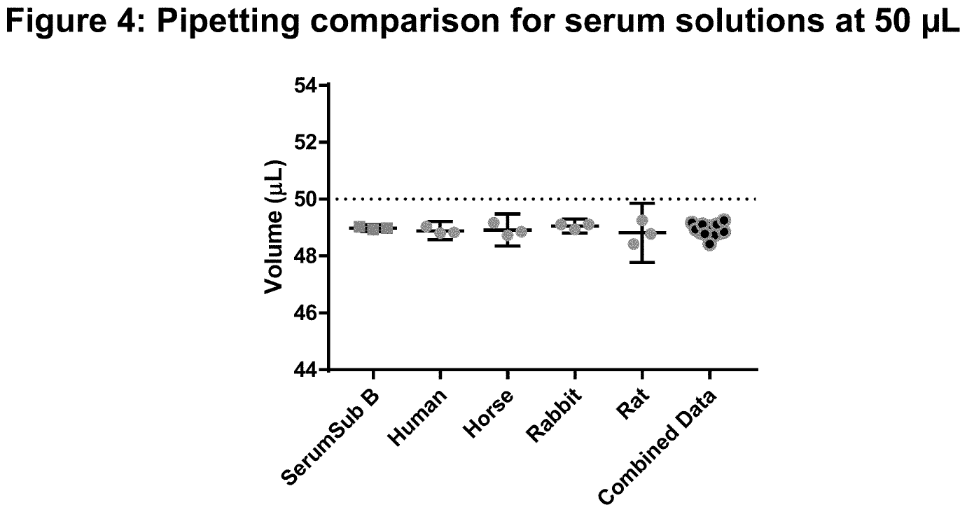

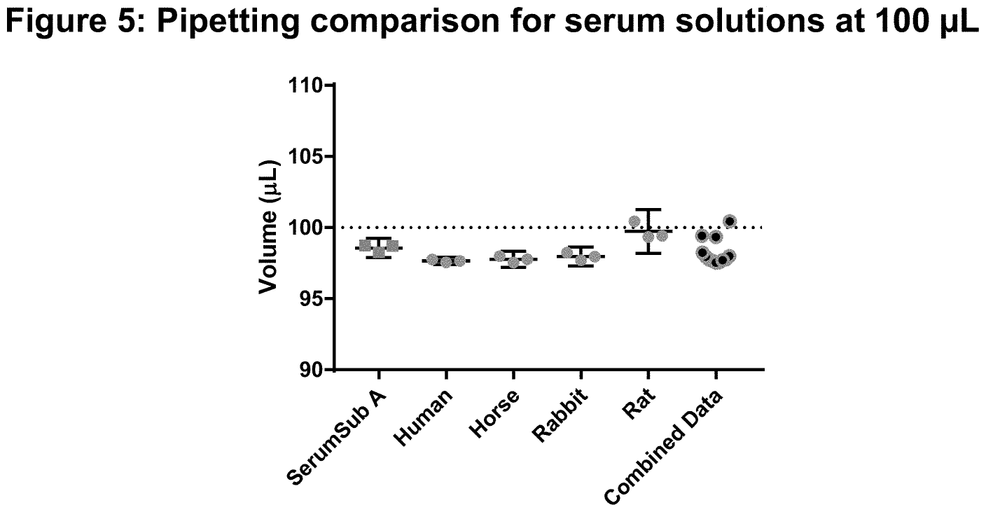

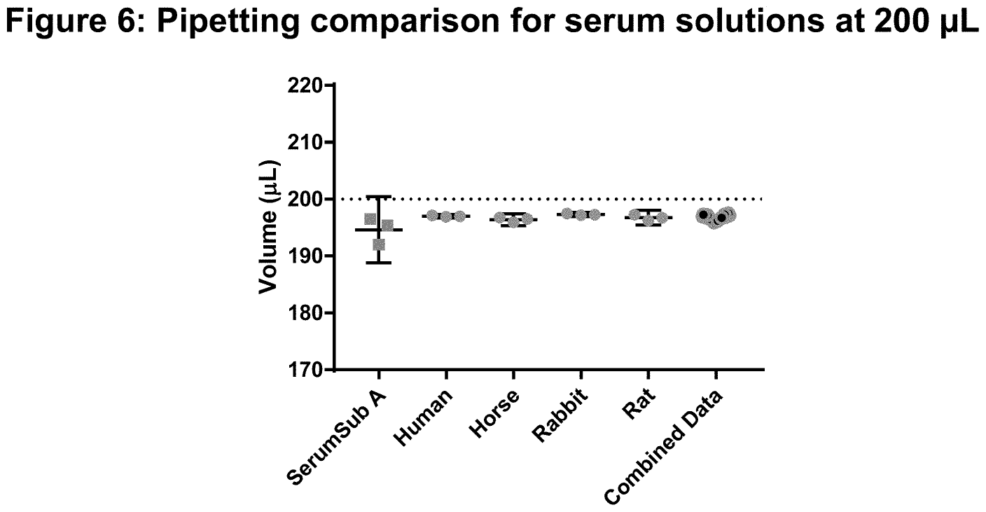

The test data in FIG. 1 and Table 1 are useful in guiding which additives might be introduced to improve the rheological properties. This data proves a useful guide. However, the more important parameter to match is the pipettability of the replacement fluids. FIGS. 2-6 demonstrate pipetted volumes of various fluids including human serum, horse serum, rabbit serum, rat serum. Also included are the pipetting data for a first example of a replacement serum fluid (SerumSub A) and for a second example of a replacement serum fluid (SerumSub B). Adjusting the rheological properties is important, but only to the extent that the desired pipettability is achieved. For example, the viscosity as a function of shear rate of the SerumSub A and SerumSubB, shown in FIG. 1, are not identical to either the human or horse serum. However, they are close enough to provide a good replacement fluid for mimicking pipetting properties. This can be observed in FIGS. 2-6 by how closely the pipetted volumes of SerumSub A and SerumSub B match those same volumes for the various sera.

The present invention further includes a related method for replacing a target fluid for pipetting activities with a replacement fluid that functions substantially the same as the target fluid in terms of pipetting characteristics. A first step of the method is to determine pipetting characteristics for the target fluid. A second step is to identify one or more primary equivalent fluids that may substantially match the pipettability of the target fluid based on the identified pipetting characteristics of the target fluid. A third step is to analyze one or more rheological and/or other properties of the one or more identified primary equivalent fluids and compare them to the corresponding properties of the target fluid. The identified primary equivalent fluid(s) should be less expensive and/or more readily available as compared to the target fluid. A fourth step is optionally to introduce one or more additives to the primary equivalent fluid or fluids and evaluate adjustments made to the pipettability of the primary equivalent fluid(s) to determine mimicking of target fluid pipettability. The one or more additives may include pipettability-modifying and non-modifying additives. A fifth step of the method is to replace the target fluid with one or more selected ones of the identified primary equivalent fluids. An optional step is to carry out analysis of the primary equivalent fluid, which may include one or more spectroscopic analyses, wherein the primary equivalent fluid may include a non-modifying additive for that purpose.

While the invention has been described with respect to specific example embodiments, it is not intended to be limited to those specific embodiments. Instead, the invention covers those embodiments and all reasonable equivalents.

What is claimed is:

1. A replacement fluid suitable to replace a target fluid, wherein the replacement fluid is different from the target fluid, the replacement fluid comprising:
a fluid having pipetting characteristics substantially equivalent to the target fluid, wherein for a desired pipetted volume of the target fluid delivered by a pipette, a pipetted volume of the replacement fluid delivered by the pipette is substantially similar to the desired pipetted volume of the target fluid.

2. The replacement fluid as claimed in claim 1, wherein the pipetted volume of the replacement fluid is within about +/−20 of the desired pipetted volume of the target fluid.

3. The replacement fluid as claimed in claim 1, wherein the replacement fluid has rheological and other properties substantially equivalent to rheological and other properties of the target fluid.

4. The replacement fluid as claimed in claim 3, wherein the rheological and other properties include viscosity, shear rate, surface tension, and contact angle.

5. The replacement fluid as claimed in claim 4, wherein the replacement fluid includes a primary equivalent fluid and one or more additives.

6. The replacement fluid as claimed in claim 5, wherein the one or more additives includes an additive that adsorbs to a surface of a pipette tip.

7. The replacement fluid as claimed in claim 5, wherein the one or more additives include one or more pipetting-modifying additives selected to modify one or more of the rheological and other properties of the primary equivalent fluid.

8. The replacement fluid as claimed in claim 7, wherein the one or more pipetting-modifying additives are selected from the group consisting of water, a water-soluble polymer, buffering salt, glycerol, sucrose, protein, lipid, and surfactant.

9. The replacement fluid as claimed in claim 5, wherein the one or more additives include one or more non-pipetting-modifying additives selected not to modify one or more of the rheological properties of the primary equivalent fluid.

10. The replacement fluid as claimed in claim 9, wherein the one or more non-pipetting-modifying additives are selected from the group consisting of stabilizing buffer, chelator, and preservative agent.

11. The replacement fluid as claimed in claim 8, wherein the replacement fluid is water and one of the one or more pipetting-modifying additives is bovine serum albumin.

12. A replacement fluid suitable to replace a target fluid to be analyzed, wherein the replacement fluid is different from the target fluid, the replacement fluid comprising:
a fluid having pipetting characteristics substantially equivalent to the target fluid, wherein for a desired pipetted volume of the target fluid delivered by a pipette, a pipetted volume of the replacement fluid delivered by the pipette is substantially similar to the desired pipetted volume of the target fluid; and
one or more analysis-aiding components, wherein the one or more analysis-aiding components are selected to enable analysis of the replacement fluid as though it were the target fluid.

13. The replacement fluid as claimed in claim 12, wherein the one or more analysis-aiding components are one or more absorbance or fluorescence dyes selected to enable spectroscopic analysis of the replacement fluid.

14. The replacement fluid as claimed in claim 12, wherein the replacement fluid has rheological and other properties substantially equivalent to rheological properties of the target fluid.

15. The replacement fluid as claimed in claim 14, wherein the rheological and other properties include viscosity, shear rate, surface tension, and contact angle.

16. The replacement fluid as claimed in claim 15, wherein the replacement fluid includes a primary equivalent fluid and one or more additives.

17. The replacement fluid as claimed in claim 16, wherein the one or more additives include one or more pipetting-modifying additives selected to modify one or more of the rheological and other properties of the primary equivalent fluid.

18. The replacement fluid as claimed in claim 17, wherein the one or more pipetting-modifying additives are selected from the group consisting of water, buffering salt, glycerol, sucrose, protein, lipid, water-soluble polymer, and surfactant.

19. The replacement fluid as claimed in claim 16, wherein the one or more additives include one or more non-pipetting-modifying additives selected not to modify one or more of the rheological properties of the primary equivalent fluid.

20. The replacement fluid as claimed in claim 19, wherein the one or more non-pipetting-modifying additives are selected from the group consisting of stabilizing buffer, chelator, and preservative agent.

21. The replacement fluid as claimed in claim 18, wherein the replacement fluid is water and one of the one or more pipetting-modifying additives is bovine serum albumin.
